SEQUENCE LISTING (4 sequences)

SEQ ID NO: 1 - RNA, 22 nt, Homo sapiens
  source 1..22: mol_type = other RNA
taacactgtctggtaacgatgt

SEQ ID NO: 2 - RNA, 22 nt, Homo sapiens
  source 1..22: mol_type = other RNA
taatactgcctggtaatgatga

SEQ ID NO: 3 - RNA, 21 nt, synthetic construct
  source 1..21: mol_type = other RNA
  misc_feature 1..21: note = for use as a negative control
  modified_base 20..21: mod_base = OTHER; note = thymine
ttctccgaacgtgtcacgttt

SEQ ID NO: 4 - RNA, 21 nt, synthetic construct
  source 1..21: mol_type = other RNA
  misc_feature 1..21: note = for use as a negative control
  modified_base 20..21: mod_base = OTHER; note = thymine
acgtgacacgttcggagaatt

(19) United States
(12) Patent Application Publication
(10) Pub. No.: US 2024/0209365 A1
(43) Pub. Date: Jun. 27, 2024

(54) METHOD FOR IMPROVING DIABETIC WOUND HEALING USING MIR-200B

(71) Applicant: Hungkuang University, Taichung City (TW)

(72) Inventor: Wan-Yu Lo, Taichung City (TW)

(21) Appl. No.: 18/391,648

(22) Filed: Dec. 20, 2023

(30) Foreign Application Priority Data
Dec. 27, 2022 (TW) 111150218

(51) Int. Cl. C12N 15/113; A61K 9/127; A61K 31/7105; A61P 17/02

(52) U.S. Cl. CPC C12N 15/113; A61K 9/127; A61K 31/7105; A61P 17/02; C12N 2310/141

(57) ABSTRACT

A method for improving diabetic wound healing includes administering to a subject in need thereof a pharmaceutical composition including microRNA-200b (miR-200b). The miR-200b has a nucleotide sequence of SEQ ID NO: 2. The subject has a diabetic ulcer. The pharmaceutical composition further includes a pharmaceutically acceptable carrier and is administered by a route selected from the group consisting of parenteral administration and topical administration.

### CROSS-REFERENCE TO RELATED APPLICATION

This application claims priority to Taiwanese Invention Patent Application No. 111150218, filed on Dec. 27, 2022, and incorporated by reference herein in its entirety.

### SEQUENCE LISTING XML

The Sequence Listing submitted concurrently herewith with a file name of “PE-69151-AM-SEQUENCE LISTING.xml,” a creation date of Nov. 14, 2023, and a size of 6.11 kilobytes, is part of the specification and is incorporated by reference in its entirety.

### FIELD

The present disclosure relates to a method for improving diabetic wound healing using a pharmaceutical composition including microRNA-200b (miR-200b).

### BACKGROUND

Patients with diabetes mellitus frequently experience a prolonged wound healing process or the wounds in these patients fail to achieve complete closure, increasing a risk of diabetic ulcer, such as diabetic foot ulcer.

Although various methods, such as applying antibiotic ointments (e.g., Flamazine® and Silvadene®), or dressings (e.g., collagen dressings and alginate dressings) on the wounds, are currently employed clinically to improve wound healing, the aforesaid methods are not ideal for the treatment of diabetic wounds, and may cause serious side effects and adverse effects.

MicroRNA (miRNA) is a short fragment of RNA molecule found in eukaryotic cells and plays a crucial role in post-transcriptional gene regulation. Previous studies have demonstrated that miRNA may be involved in the pathogenesis of diabetes mellitus. For example, it has been reported in Filios S. R. et al. (2014), J. Biol. Chem., 289:36275-36283 that microRNA-200 (miR-200) family, including microRNA-200a (miR-200a), microRNA-200b (miR-200b), and microRNA-200c (miR-200c), is abundantly expressed in diabetic mice and may further induce pancreatic β-cell apoptosis, leading to a worsening of diabetes.

In spite of the aforesaid, there is still a need to develop an effective way for improving diabetic wound healing.

### SUMMARY

Therefore, an object of the present disclosure is to provide a method for improving diabetic wound healing, which can alleviate at least one of the drawbacks of the prior art, and which includes administering to a subject in need thereof a pharmaceutical composition including microRNA-200b (miR-200b).

The miR-200b has a nucleotide sequence of SEQ ID NO: 2.

### DETAILED DESCRIPTION

For the purpose of this specification, it will be clearly understood that the word “comprising” means “including but not limited to”, and that the word “comprises” has a corresponding meaning.

It is to be understood that, if any prior art publication is referred to herein, such reference does not constitute an admission that the publication forms a part of the common general knowledge in the art, in Taiwan or any other country.

Unless defined otherwise, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The present disclosure provides a method for improving diabetic wound healing, which includes administering to a subject in need thereof a pharmaceutical composition including microRNA-200b (miR-200b).

As used herein, the term “wound” refers to various wounds including, but is not limited to, chronic wound (such as diabetic ulcer, venous ulcer, arterial ulcer, pressure ulcer, infectious ulcer, gastrointestinal ulcer, surgical wound, and burn) and acute wound (such as cut, scrape, and laceration). In certain embodiments, the wound may be the diabetic ulcer.

As used herein, the term “wound healing” can be used interchangeably with other terms such as “wound repair.”

As used herein, the term “administration” or “administering” means introducing, providing or delivering a pre-determined active ingredient to a subject by any suitable routes to perform its intended function.

As used herein, the term “subject” refers to any animal of interest, such as humans, monkeys, cows, sheep, horses, pigs, goats, dogs, cats, mice, rats, and fish. In certain embodiments, the subject is a human.

In certain embodiments, the subject suffers from diabetes mellitus and has a diabetic ulcer.

According to the present disclosure, the diabetes mellitus includes type 1 diabetes mellitus (T1DM) and type 2 diabetes mellitus (T2DM). In certain embodiments, the diabetes mellitus may be T2DM.

As used herein, the term “miR-200b” refers to diverse forms of miR-200b, such as single stranded, double stranded, or partially double stranded of mature miRNA, precursor miRNA (pre-miRNA), primary miRNA (pri-miRNA), and miRNA mimic.

According to the present disclosure, the miR-200b suitable for use in this disclosure may be obtained as commercial products, or may be prepared using methods well-known to those skilled in the art. Examples of the methods may include, but are not limited to, chemical synthesis, in vitro transcription, in vivo expression, and combinations thereof.

In some embodiments, the miR-200b may have a nucleotide sequence corresponding to nucleotide residues 57 to 78 of the nucleotide sequence having NCBI Accession Number NR_029639.1.

In an exemplary embodiments, the miR-200b has a nucleotide sequence of SEQ ID NO: 2.

According to the present disclosure, the pharmaceutical composition may further include a pharmaceutically acceptable carrier widely employed in the art of drug-manufacturing. For instance, the pharmaceutically acceptable carrier may include one or more of the following agents: solvents, buffers, emulsifiers, suspending agents, decomposers, disintegrating agents, dispersing agents, binding agents, excipients, stabilizing agents, chelating agents, diluents, gelling agents, preservatives, wetting agents, lubricants, absorption delaying agents, liposomes, and the like. The choice and amount of the aforesaid agents are within the expertise and routine skills of those skilled in the art.

According to the present disclosure, the pharmaceutical composition may be formulated into a dosage form suitable for parenteral or topical administration using technology well known to those skilled in the art.

For parenteral administration, the pharmaceutical composition according to the present disclosure may be formulated into an injection, e.g., a sterile aqueous solution or a dispersion.

The pharmaceutical composition according to the present disclosure may be administered via one of the following parenteral routes: intraperitoneal injection, intrapleural injection, intramuscular injection, intravenous injection, intraarterial injection, intraarticular injection, intrasynovial injection, intrathecal injection, intracranial injection, intraepidermal injection, subcutaneous injection, intradermal injection, intralesional injection, and sublingual administration. In certain embodiments, the pharmaceutical composition may be administered via subcutaneous injection.

According to the present disclosure, the pharmaceutical composition may be formulated into an external preparation suitable for topical application to the skin using technology well known to those skilled in the art. The external preparation includes, but is not limited to, emulsions, gels, ointments, creams, patches (e.g., microneedle patch, MNP), liniments, powders, aerosols, sprays, lotions, serums, pastes, foams, drops, suspensions, salves, and bandages.

According to the present disclosure, the dose and frequency of administration of the pharmaceutical composition may vary depending on the following factors: the severity of the illness or disorder to be treated, routes of administration, and age, physical condition and response of the subject to be treated. In general, the pharmaceutical composition may be administered in a single dose or in several doses.

The disclosure will be further described by way of the following examples. However, it should be understood that the following examples are solely intended for the purpose of illustration and should not be construed as limiting the disclosure in practice.

### EXAMPLES

### General Experimental Materials:

1. MicroRNAs (miRNAs)

A microRNA-200a (miR-200a), a microRNA-200b (miR-200b), and two miR-NCs (i.e., miR-NC1 and miR-NC2 serving as negative controls) used in the following experiments were all synthesized as miRNA mimics by GeneDireX, Inc.

The detailed information regarding each of the four miRNAs are listed in Table 1 below.

TABLE 1

| | | Nucleotide sequence |
|---|---|---|
| MiRNA | | (5′→3′) |
| MiR-200a | | uaacacugucugguaacgaugu |
| NCBI accession no. NR_029834.1) | | (SEQ ID NO: 1) |
| MiR-200b | | uaauacugccugguaaugauga |
| (NCBI accession no. NR_029639.1) | | (SEQ ID NO: 2) |
| MiR-NC | MiR-NC1 | uucuccgaacgugucacgutt |
| | | (SEQ ID NO: 3) |
| | MiR-NC2 | acgugacacguucggagaatt |
| | | (SEQ ID NO: 4) |

Prior to the following experiments, each of the miR-200a and miR-200b was mixed with Lipofectamine™ 3000 (Invitrogen, Cat. no. L3000015) in a volume ratio of 1:1 to encapsulate each of the two miRNAs within the liposomes. In addition, the miR-NC1 and miR-NC2 were mixed in a volume ratio of 1:1, so as to obtain a miR-NC mixture, followed by mixing the miR-NC mixture with the Lipofectamine™ 3000 in a volume ratio of 1:1 to encapsulate the miR-NC mixture within the liposomes. Each of the resultant liposome-encapsulated miRNA was added to a 20-fold volume of physiological saline, so as to obtain three miRNA-liposome solutions, i.e., a miR-200a-liposome solution, a miR-200b-liposome solution, and a miR-NC-liposome solution.

### 2. Experimental Mice

Male db/db mice (12 weeks old, with a body weight of approximately 46.44±1.159 g) used in the following experiments were purchased from National Laboratory Animal Center, R.O.C. All the experimental mice were housed in an animal room with an independent air conditioning system under the following laboratory conditions: an alternating 12-hour light and 12-hour dark cycle, a temperature maintained at 22±3° C., and a relative humidity maintained at 60% to 70%. The mice were provided with water and fed ad libitum. All experimental procedures involving the experimental mice were in compliance with the legal provision of the Institutional Animal Care and Use Committee (IACUC) of Hungkuang University, Taiwan, and were carried out according to the guidelines of IACUC Animal Experiment no. NK-11111.

### General Procedures:

### 1. Statistical Analysis

All the experiments described below were performed in triplicates. The experimental data of all the test groups are expressed as mean±standard error of the mean (SEM), and were analyzed using student's t-test, so as to evaluate the differences between the groups. Statistical significance is indicated by p<0.05.

### Example 1. Evaluation of the Effect of Different miRNAs on Improvement of Diabetic Wound Healing

### Experimental Procedures:

A. Induction of Diabetic Wound and Administration of miRNA

First, the male db/db mice as described in section 2 of “General Experimental Materials” were randomly divided into 3 groups, including a comparative group (n=5), an experimental group (n=7), and a pathological control group (n=5). Next, each mouse was anesthetized with 3% to 4% isoflurane (Panion Bf Biotech Inc., Cat. no. DP4900V22LA01), and then a back of each mouse was shaved to expose a dorsal epidermis and disinfected three times with 75% alcohol (Echo Chemical Co., Ltd., Cat. no. 18000135-1). Afterwards, the dorsal epidermis of each mouse was punched with an 8 mm biopsy punch (Kai Medical, Cat. no. BP-80F) to create a circular wound with an area of approximately 50 mm² to 70 mm². Subsequently, the circular wound of each mouse was immediately injected with nalbuphine (serving as an analgesic, Uni Pharma Co., Ltd., Cat. no. BKH381) at a dose of 0.5 mg/100 g body weight, followed by application of a waterproof breathable dressing (3M™ Tegaderm™, St. Paul, MN, Cat. no. 7000002870) for 24 hours to prevent the circular wound from being infected.

After removing the waterproof breathable dressings, the circular wound of the respective mouse of each group was divided into four areas (i.e., upper, lower, left, and right areas of the circular wound), which were then subcutaneously injected with the corresponding miRNA-liposome solution prepared in section 1 of “General Experimental Materials”, as shown in Table 2. The total dose for the four areas was 100 ng (i.e., 100 ng per circular wound).

TABLE 2

| Group | Treatment agent |
|---|---|
| Pathological control group | MIR-NC-liposome solution |
| Comparative group | MIR-200a-liposome solution |
| Experimental group | MIR-200b-liposome solution |

### B. Determination of Wound Area

Before the start of the miRNA-liposome solution administration (i.e., on the 0^{th} day) and on the 13^{th} day after the start of the miRNA-liposome solution administration, the circular wound of each mouse was photographed, followed by subjecting the thus obtained images to determination of wound area using an ImageJ software. Then, the percentage of wound area for each group was calculated by substituting the thus obtained mean wound area for each group determined on the 0^{th} day after the start of the miRNA-liposome solution administration and that determined on the 13th day after the start of the miRNA-liposome solution administration into the following Equation (1):

$$A = ( B / C ) \times 100 \% \qquad ( 1 )$$

$$\begin{matrix}where A = percentage of wound area ( \% ) \\ B = mean wound area determined on the 13 th day after start of the miRNA ‐ liposome solution administration \\ C = mean wound area determined on the 0 th day after start of the miRNA ‐ lipsome solution administration\end{matrix}$$

The data thus obtained were analyzed according to the procedures as described in section 1 of “General Procedures.” The results are shown in Table 3 below.

### Results:

TABLE 3

| Group | Percentage of wound area (%) |
|---|---|
| Pathological control group | 88.7 ± 6.8 |
| Comparative group | 92.7 ± 9.3 |
| Experimental group | 49.5 ± 11.2** |

Note:
The symbol “**” represents p < 0.01 (compared with the pathological control group).

As shown in Table 3, the percentage of wound area determined in the comparative group was not significantly different from that determined in the pathological control group, whereas the percentage of wound area determined in the experimental group was significantly lower than those determined in the pathological control group and the comparative group. These results demonstrate that the miR-200b can effectively improve diabetic wound healing, and hence is capable of alleviating diabetic ulcer.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for improving diabetic wound healing, comprising administering to a subject in need thereof a pharmaceutical composition including microRNA-200b (miR-200b),
wherein the miR-200b has a nucleotide sequence of SEQ ID NO: 2.

2. The method as claimed in claim 1, wherein the subject has a diabetic ulcer.

3. The method as claimed in claim 1, wherein the pharmaceutical composition further includes a pharmaceutically acceptable carrier.

4. The method as claimed in claim 1, wherein the pharmaceutical composition is administered by a route selected from the group consisting of parenteral administration and topical administration.
